(19) United States
(12) Patent Application Publication
(10) Pub. No.: US 2023/0386662 A1
(43) Pub. Date: Nov. 30, 2023

(54) RAPID AND DIRECT IDENTIFICATION AND DETERMINATION OF URINE BACTERIAL SUSCEPTIBILITY TO ANTIBIOTICS

(71) Applicants: B. G. NEGEV TECHNOLOGIES AND APPLICATIONS LTD., AT BEN-GURION UNIVERSITY, Beer Sheva (IL); SAMI SHAMOON COLLEGE OF ENGINEERING, Beer Sheva (IL); AFEKA, TEL AVIV COLLEGE OF ENGINEERING, Tel Aviv (IL)

(72) Inventors: Mahmoud HULEIHEL, Beer Sheva (IL); Ahmad SALMAN, Beer Sheva (IL); Itshak LAPIDOT, Beer Sheva (IL)

(21) Appl. No.: 18/032,617

(22) PCT Filed: Oct. 19, 2021
(86) PCT No.: PCT/IL2021/051237

§ 371 Date: Apr. 19, 2023

Related U.S. Application Data
(60) Provisional application No. 63/093,429, filed on Oct. 19, 2020.

(51) Int. Cl. G16H 50/20; G16B 15/30; G16B 40/20; G16H 70/60; G01N 21/3577; G01N 33/487

(52) U.S. Cl. CPC G16H 50/20; G16B 15/30; G16B 40/20; G16H 70/60; G01N 21/3577; G01N 33/487; G01N 2021/3595

(57) ABSTRACT

A method comprising: receiving spectral data associated with each of a plurality of bodily fluid samples obtained from a corresponding plurality of subjects having a specified type of infectious disease; receiving data identifying a response parameter to one or more of a set of therapies associated with each of the subjects; at a training stage, training a machine learning model on a training set comprising: (i) the spectral data associated with each of the plurality of bodily fluid samples, and (ii) labels associated with the response parameters; and at an inference stage, applying the trained machine learning model to target spectral data associated with a target bodily fluid sample obtained from a target subject, to estimate a response in the target subject to each specified therapy in the set of specified therapies.

### CROSS-REFERENCE TO RELATED APPLICATIONS

This application claims the benefit of priority to U.S. Provisional Application No. 63/093,429 filed on Oct. 19, 2020, titled “RAPID AND DIRECT IDENTIFICATION AND DETERMINATION OF URINE BACTERIAL SUSCEPTIBILITY TO ANTIBIOTICS”, the contents of which are incorporate herein by reference in their entirety.

### FIELD OF THE INVENTION

The present invention relates to the field of machine learning.

### BACKGROUND

One of the major human bacterial infections are urinary tract infections (UTIs), which are caused mainly (80%-95%) by Escherichia (E.) coli, Klebsiella pneumoniae and Pseudomonas aeruginosa. Antibiotics are considered as the most effective treatment for bacterial infections. However, most bacteria already have developed resistance to the most of commonly available antibiotics, resulting in difficult-to-treat infections. Therefore, it is crucial to determine the susceptibility of the infecting bacterium to antibiotic for prescribing effective treatment. Known methods are time-consuming as they require approx. 48 hours for determining bacterial susceptibility.

Thus, it is highly important to develop new objective methods that can significantly reduce the time required to determine the bacterial susceptibility to antibiotics.

The foregoing examples of the related art and limitations related therewith are intended to be illustrative and not exclusive. Other limitations of the related art will become apparent to those of skill in the art upon a reading of the specification and a study of the figures.

### SUMMARY OF INVENTION

The following embodiments and aspects thereof are described and illustrated in conjunction with systems, tools and methods which are meant to be exemplary and illustrative, not limiting in scope.

There is provided, in an embodiment, a system comprising at least one hardware processor; and a non-transitory computer-readable storage medium having stored thereon program instructions, the program instructions executable by the at least one hardware processor to: receive spectral data associated with each of a plurality of bodily fluid samples obtained from a corresponding plurality of subjects having a specified type of infectious disease, receive data identifying a response parameter to one or more of a set of therapies associated with each of the subjects, at a training stage, train a machine learning model on a training set comprising: (i) the spectral data associated with each of the plurality of bodily fluid samples, and labels associated with the response parameters, and at an inference stage, apply the trained machine learning model to target spectral data associated with a target bodily fluid sample obtained from a target subject, to estimate a response in the target subject to each specified therapy in the set of specified therapies.

There is also provided, in an embodiment, a method comprising: receiving spectral data associated with each of a plurality of bodily fluid samples obtained from a corresponding plurality of subjects having a specified type of infectious disease; receiving data identifying a response parameter to one or more of a set of therapies associated with each of the subjects; at a training stage, training a machine learning model on a training set comprising: (i) the spectral data associated with each of the plurality of bodily fluid samples, and (ii) labels associated with the response parameters; and at an inference stage, applying the trained machine learning model to target spectral data associated with a target bodily fluid sample obtained from a target subject, to estimate a response in the target subject to each specified therapy in the set of specified therapies.

There is further provided, in an embodiment, a computer program product comprising a non-transitory computer-readable storage medium having program instructions embodied therewith, the program instructions executable by at least one hardware processor to: receive spectral data associated with each of a plurality of bodily fluid samples obtained from a corresponding plurality of subjects having a specified type of infectious disease; receive data identifying a response parameter to one or more of a set of therapies associated with each of the subjects; at a training stage, train a machine learning model on a training set comprising: (i) the spectral data associated with each of the plurality of bodily fluid samples, and (ii) labels associated with the response parameters; and at an inference stage, apply the trained machine learning model to target spectral data associated with a target bodily fluid sample obtained from a target subject, to estimate a response in the target subject to each specified therapy in the set of specified therapies.

In some embodiments, the with respect to each of the bodily fluid samples, the spectral data is acquired less than 5 hours from a time of obtaining of the bodily fluid sample.

In some embodiments, the plurality of bodily fluid samples and the target sample are each a urine sample, and the specified type of infectious disease is urinary tract infection (UTI).

In some embodiments, the spectral data is acquired from bacteria obtained from each of the bodily fluid samples.

In some embodiments, the spectral data represents infrared (IR) absorption in the bacteria.

In some embodiments, the spectral data is within the wavenumber range of 600-4000 cm⁻¹.

In some embodiments, the set of therapies comprises one or more antibiotics.

In some embodiments, the response parameter in one of: sensitive and resistant.

In some embodiments, the bodily fluids comprise one of: whole blood, blood plasma, blood serum, lymph, urine, saliva, semen, synovial fluid, and spinal fluid.

In some embodiments, the program instructions are further executable to perform, and the method further comprises performing, one of: feature manipulations and dimensionality reduction with respect to the spectral data.

In some embodiments, with respect to the training set, the spectral data associated with each of the plurality of bodily fluid samples are labeled with the labels.

In some embodiments, the training set further comprises, with respect to at least some of the subjects, labels associated with clinical data.

In addition to the exemplary aspects and embodiments described above, further aspects and embodiments will become apparent by reference to the figures and by study of the following detailed description.

### BRIEF DESCRIPTION OF THE FIGURES

FIG. 1 is a flowchart of the functional steps in a process for training a machine learning model to determine the susceptibility of the infecting bacteria in urine samples of UTI patients to antibiotic, according to some embodiments of the present disclosure;

FIG. 2 shows the average IR absorption spectra of E. coli, Klebsiella pneumonia, Pseudomonas aeruginosa and other UTI bacteria in the 900-1800 cm⁻¹ region;

FIG. 3 shows the calculated SNR of 20 different isolates. It can be seen that the SNR is ˜100, which is relatively high;

FIG. 4A shows 12 spectra of one E. coli isolate, acquired from different sites of the same sample in the 900-1800 cm⁻¹ after preprocessing;

FIG. 4B shows the averages of three infrared spectra of the same isolate from three different preparations (spots);

FIG. 4C shows the averages of three infrared spectra of the same isolate measured from the same spot at three different days;

FIG. 5 shows the receiver-operating characteristic (ROC) curves of the classifier qSVM for the classification among E. coli, Klebsiella pneumonia, Pseudomonas aeruginosa and other UTI bacteria;

FIGS. 6A-6B present the average second derivative IR spectra of E. coli, in the 900-1800 cm⁻¹ region grouped as sensitive or resistant to: Amoxicillin (panel a), Ampicillin (panel c), Ceftazidime (panel e), and Ceftriaxone (panel g);

FIGS. 7A-7B present the average second derivative IR spectra of Klebsiella pneumonia, in the 900-1800 cm⁻¹ region grouped as sensitive of resistant to: Amoxicillin (panel a), Ceftazidime (panel c), Ceftriaxone (panel e) and Cefuroxime (panel g); and

FIGS. 8A-8B present the average second derivative IR spectra of Pseudomonas aeruginosa, in the 900-1800 cm⁻¹ region grouped as sensitive of resistant to: Ceftazidime (panel a), Ciprofloxacin (panel c), Gentamicin (panel e), and Imipenem (panel g).

### DETAILED DESCRIPTION

Disclosed are a system, method, and computer program product which provide for a machine-learning model configured to predict a response in a patient having an infectious disease to one or more specified therapies.

The present disclosure will discuss extensively with respect to response prediction to antibiotics in the context of patients having UTI. However, the present method may be equally effective in estimating patient response to therapy with respect to a range of bacterial infections, based on infrared absorption spectra of bacterial samples purified from bodily fluid samples obtained from the patients.

In some embodiments, the present disclosure provides for estimating a response in a subject having an infectious disease, e.g., UTI bacteria, to one or more specified antibiotics.

The present disclosure provides for a reliable, fast, and cost-effective method, which could be used as a tool by a physician to determine the effectiveness of one or more therapies (e.g., antibiotics) for targeting an infecting UTI bacterium. This may eliminate or reduce the prescribing of ineffective treatment, and thus help to decrease the development of multi-resistant bacteria. In some embodiments, response prediction and/or estimation according to the present disclosure may be obtained with respect to samples which have not undergone any culturing or multiplication or proliferation of bacteria in samples, e.g., over a period of 24 or 48 hours, or have undergone a culturing or multiplication or proliferation of less than 5 hours.

Infectious diseases caused by bacterial pathogens are considered as one of the leading reasons for serious infectious diseases that cause mortality among humans and animals. Currently, antibiotics are the most effective treatment for bacterial infections, however, overprescribing of antibiotics for treatment of infections is one of the major driving force behind the development and spread of multidrug resistant bacteria in both humans and animals.

The development of multidrug resistant bacteria has become a severe global health problem, because different bacteria have already acquired resistance to various antibiotics, and a few have become resistant to all antibiotics. Resistance to antibiotics is caused by different molecular mechanisms, such as genetic material exchange between bacteria and specific mutations. The increase in bacterial resistance to antibiotics may cause a return to the pre-antibiotics period, where it will be difficult to treat many routine infections. It was reported that 10-30% of patients with various blood infections in intensive care units do not get the appropriate antibiotic treatment at their arrival, resulting in a death rate 30-60% higher as compared with patients treated with an effective antibiotic.

Thus, rapid detection and identification of bacterial susceptibility to antibiotic is critical for effective treatment that may save lives and dramatically reduces the costs associated with inadequate treated. Currently, the methods used for determining bacterial susceptibility to antibiotics are divided into phenotypic and genotypic methods. Phenotypic methods are routinely used in medical centers require at least 48 hours for the identification of an infection as either bacterial or viral, and for the determination of its susceptibility to antibiotics. Genotypic methods for bacterial detection and susceptibility determination are not in routine use by medical centers, mainly due to their high costs.

A potential advantage of the present disclosure is, therefore, in that it provides for a rapid and reliable identification of the infecting bacterium at the species level, and determination of the UTI bacterial susceptibility to antibiotic, when the bacterial sample is purified directly from a subject's urine. Thus, it provides a non-invasive, low risk, and inexpensive healthcare tool for the treatment of UTI diseases, which will enable a physician to prescribe the most effective antibiotic for targeting the infecting bacterium, resulting in a reduction in the use of ineffective treatment, and simultaneously controlling the development of multi-resistant bacteria.

Experimental studies reported hereinbelow show that the biochemical changes in bacterial genome associated with developing resistivity are minute, and this is reflected in minor spectral changes among resistant and sensitive isolates in each of the investigated types (E. coli, Klebsiella pneumonia, and Pseudomonas aeruginosa). Previous studies have shown that acquiring antibiotic resistivity may be the result of genetic changes in the bacteria strains and the exchanging of genetic or/and chromosomal material among bacteria or via transposons and plasmids. Thus, the spectral differences based on the susceptibility of the isolates as sensitive and resistant are expected to be minor.

The spectral differences between sensitive and resistant isolates for specific antibiotic are spread over the entire spectral region (900-1800 cm⁻¹), thus, it is almost impossible to point out the exact biochemical changes, which are associated with the resistivity. Nonetheless, the differentiation between the resistant and sensitive isolates of UTI bacteria to antibiotics, which is the main aim of the current work, is the most important issue for physicians. As disclosed hereinbelow, analyzing IR absorption spectra of specified bacteria (E. coli, Klebsiella pneumoniae and Pseudomonas aeruginosa), shows a great potential of the proposed method for taxonomic classification of the most common UTI bacteria with 97% success rate.

One of the characteristics of infrared microscopy is its high sensitivity in monitoring subtle molecular changes that enable to monitor the subtle differences between the resistant and the sensitive isolates of the tested UTI bacteria (E. coli, Klebsiella pneumoniae, and Pseudomonas aeruginosa). Although these spectral differences are very small, they are repeatable and enable machine learning classifiers to achieve promising classification performance, as shown by the present inventors in experimental results reported hereinbelow.

Specifically, Fourier-transform infrared (FTIR) spectroscopy is a powerful tool for biochemical analysis, and can provide detailed information about chemical composition at the molecular level. FTIR has high sensitivity, high resolution, high signal-to-noise ratio (SNR), and is simple and cost-effective to use. Infrared (IR) microscopy has advanced significantly, with improved spectral and spatial resolutions, making it is possible to acquire unprecedented biochemical information at the molecular level for cells (both prokaryotic and eukaryotic). For example, IR spectroscopy is able to detect minor molecular changes, such as early changes during the development of diseases or cell transformation at a stage when the morphology is still normal. Thus, FTIR spectroscopy provides a powerful tool for biochemical analysis, with the ability to distinguish among a wide range of biomolecules based on spectral signatures in the mid-IR absorption range (i.e., wavenumbers in the range of 600-4000 cm⁻¹).

Accordingly, in some embodiments, the present disclosure provides for FTIR spectroscopy to determine UTI bacterial susceptibility to therapy.

In some embodiments, the present disclosure provides for training a machine learning model, using a training dataset comprising a plurality of bacterial samples obtained from urine samples of a plurality of individuals. In some embodiments, a trained machine learning model of the present disclosure may provide for predicting a response of a target patient, diagnosed with a specified infectious disease, to an associated specified treatment or therapy.

In some embodiments, a training dataset for a machine learning model of the present disclosure may comprise a plurality of spectral values associated with UTI bacteria from a cohort of subjects. In some embodiments, a training dataset may be annotated with category labels denoting a response susceptibility of each bacterium to one or more associated treatments. In some embodiments, the training dataset may be annotated with category labels denoting a response susceptibility to a specified antibiotic. In some embodiments, additional and/or other annotation schemes may be employed. In some embodiments, the training dataset may further be annotated with category labels denoting, e.g., clinical data.

In some embodiments, a trained machine learning model of the present disclosure provides for predicting a response in a subject to a specified treatment or therapy as a binary value, e.g., ‘sensitive’/‘resistant,’ ‘yes/no,’ ‘responsive/non-responsive,’ or ‘favorable/non-favorable response.’ In some embodiments, the prediction may be expressed on a scale and/or be associated with a confidence parameter.

Accordingly, in some embodiments, a machine learning model of the present disclosure may provide for predicting a response rate and/or success rate of a specified treatment in a subject. For example, in some embodiments, the prediction may be expressed in discrete categories and/or on a gradual scale.

In some embodiments, spectral measurement may be obtained with respect to each bacterial sample, e.g., FTIR measurements in the 600-4000 cm⁻¹ wavenumber region.

In some embodiments, the obtained spectral data may be pre-processed to improve the spectral features, and to facilitate spectral interpretation and analysis. For example, atmospheric compensation may be applied to account for ambient humidity and CO₂ influences in each spectrum. In some embodiments, other and/or additional preprocessing methods may be applied, e.g., the spectra may be smoothed by a suitable algorithm, such as the Savitzky-Golay algorithm, to reduce high frequency instrumental noise; the spectral range may be cut, e.g., to a range of 900-1800 cm⁻¹; and/or the spectra may be baseline corrected, and vector and offset normalizations may be applied.

In some embodiments, features manipulation, feature selection and/or dimensionality reduction steps may be applied to the preprocessed spectra, to obtain a set of features providing an informative compact representation of the measured spectra. In some embodiments, the result of the feature selection and/or dimensionality reduction steps is a low-dimensional representation of the obtained spectra, which comprises selected features for use in training a machine learning model.

In some embodiments, a machine learning models of the present disclosure may then be trained on the constructed training dataset. In some embodiments, a trained machine learning models of the present disclosure may be configured for predicting of the susceptibility of a target bacterium to a specific antibiotic.

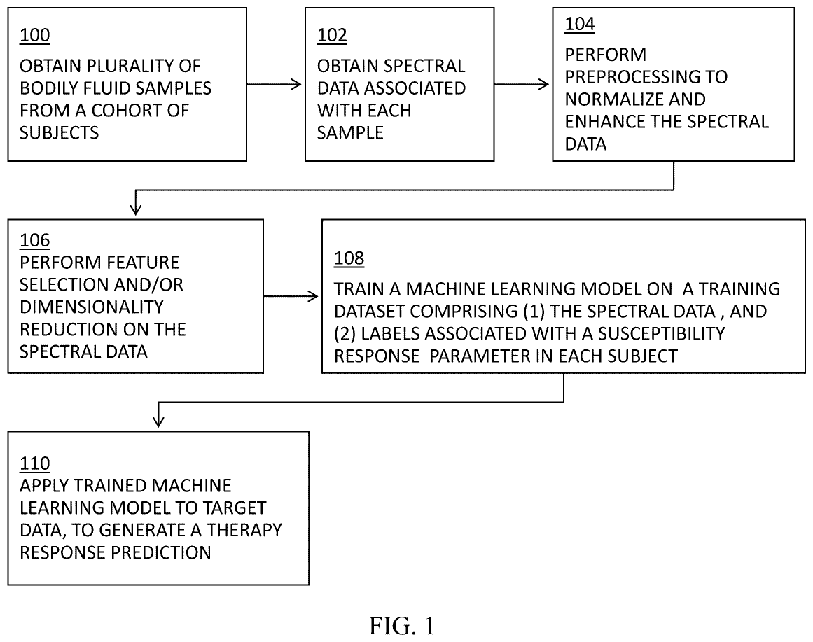

FIG. 1 is a flowchart of the functional steps in a process for training a machine learning model to determine the susceptibility of the infecting bacteria in urine samples of UTI patients to antibiotic.

In some embodiments, step 100 comprises a sample acquisition and preparation step. Accordingly, in some embodiments, at step 100, a urine sample may be obtained from each subject in a cohort of subjects diagnosed as having an UTI infectious disease. In some embodiments, infected bacteria may be identified, e.g., at the species level, in each of the samples.

In some embodiments, the samples may undergo a purification process wherein the contaminating bacteria may be isolated and purified using, e.g., a centrifuge or any suitable method. For example, about five milliliters from each sample may be centrifuged for five minutes at 1000 g, wherein the resulting pellets may be washed with double distilled water (DDW) several times in order to eliminate any nonbacterial contaminants. In some embodiments, the obtained bacteria pellets may be suspended in, e.g., 50 μl of DDW, and the concentration of the bacteria is measured using, e.g., a spectrometer.

In some embodiments, 2 μl of the resulting bacterial sample may be placed on windows transparent to mid-infrared radiations like a zinc selenide (ZnSe) slide, and air dried at room temperature for few minutes.

In some embodiments, at step 102, spectral signatures may be acquired with respect to each of the processed samples. In some embodiments, for example, spectral measurements may be performed using an FTIR spectrometer, e.g., incorporating a liquid nitrogen cooled mercury cadmium telluride (MCT) detector using the transmission mode. In some embodiments, the measurements may be performed using 128 co-added scans in the 600-4000 cm⁻¹ wavenumber region with 4 cm⁻¹ spectral resolution. In some embodiments, several spectra from different sites of the same sample are acquired. In some embodiments, each single spectrum used may be an average of several spectra measured from different sites of the same sample.

In some embodiments, at step 104, a pre-processing stage may be performed, to improve the spectral features and facilitate spectral interpretation and analysis. For example, atmospheric compensation may be applied to eliminate the ambient air humidity and CO₂ influences for each spectrum. In some embodiments, the spectra may be smoothed using, e.g., a Savitzky-Golay algorithm and/or any other suitable algorithm, to reduce the high frequency instrumental noise, and second derivative of each wavenumber may be calculated. In some embodiments, preprocessing may include, e.g., reducing the spectral range, performing baseline correction using, e.g., a Concave Rubber Band method, performing feature manipulation, and/or performing vector and offset normalizations.

In some embodiments, at step 106, a feature selection and/or dimensionality reduction step may be performed.

In some embodiments, feature selection may be performed to extract informative representation from the raw data. In some embodiments, dimensionality reduction may be performed to ensure compact representation of the data by reducing the dimensionality of the initial feature vectors. In some embodiments, such techniques as Chi-square method and/or symmetrical Kullback-Leibler (KL) divergence may be used. In some embodiments, the result of this stage is a low-dimensional representation (selected features) of the raw data.

In some embodiments, a Chi-square method computes the interdependence of two categories for each wavenumber in the data, on the second derivative categories. Then, the wavenumbers are arranged in descending order based on the Chi-square scores, with the most discriminative wavenumber (highest score) first. The optimal set of features is estimated during a nested k-fold process, by adding a specified number of features each time, and then training and testing a machine learning model on the selected features. The set that gives the best results is chosen for training the entire system.

In some embodiments, a symmetrical KL divergence method may comprise estimating, for each feature (i.e., second derivative of each wavenumber) and each classification category (e.g., resistant and sensitive), a univariate Gaussian distribution, respectively. The score is calculated according to the following expression:

S=KL(G_{S}∥G_{R})+KL(G_{R}∥G_{S})

where KL(G_{S}∥G_{R}) measures a dissimilarity of the hypothesized distribution G_{R} from the true distribution G_{S} and vice versa. The score is equal to zero only if G_{R} is equal to G_{S}, otherwise, the score is positive. For highly separated classes the score is high. Better features are those that have higher scores.

In some embodiments, preprocessing step 106 may comprise at least one of: data cleaning and normalizing, data quality control, data transformations, and/or statistical tests calculated in order to assess the data quality.

In some embodiments, at step 108, a training dataset of the present disclosure may be used to train a machine learning model, e.g., a classifier, based, e.g., on any suitable algorithm such as, but not limited to, a random forest (RF) algorithm, extreme gradient boosting (XGBoost), and/or support vector machine (SVM).

In some embodiments, XGBoost is based on first selecting a single random decision tree as a start. The algorithm may then perform multiple iterations, where each time, a new decision tree is added, such that the error is reduced as the results of the new tree are added. The end result is a set of constructed trees which constitutes the whole model. In some embodiments, the final decision is a weighted sum of the trees decisions.

In some embodiments, random forest (RF) methods are based on choosing sub-sets of features randomly from the feature vector, wherein different decision tree are designed according to these sub-sets. The category of each spectrum in the test set is predicted separately using each reduced dimension classifier (tree). The final decision is according to the majority vote over the decisions of all the trees.

In some embodiments, SVM methods are based on a discriminative classifier formally defined by a separating hyperplane. SVM is widely used because of its powerful ability of classification. When a linear classification is impossible, a kernel is applied in order to perform a linear separation on features after a non-linear transformation.

In some embodiments, the trained machine learning model may be validated on a portion of the dataset reserved for this purpose. In some embodiments, a k-fold cross validation technique may be applied, wherein the entire dataset may be divided into k disjoint folds. One of the folds is reserved for validation, while the remaining folds are used for training. The process is repeated k times, wherein each time a different fold is reserved for validating. In some embodiments, nested cross-validation methods may be used for defining the hyper-parameters of the algorithms, and/or the feature selection process.

In some embodiments, a k-fold cross-validation approach is adopted in order to validate the performances of each of the used machine learning algorithms. In some embodiments, a 5-fold approach may be used.

In the case of random forest, the algorithm is based on a collective decision of many trees. The decision logic is a majority vote, e.g., it counts how many trees return each of the category classes. When XGBoost is applied, the decision is also based on collective decision of many trees. However, it is calculated based on a confidence weight of each tree, where the final decision is a sign operator over the weighted sum of all the trees decisions. In the case of SVM, the score is positive if the sample is above the hyper-plan (indicating a first category class), or a negative value if the sample is below the hyper-plan (indicating a second category class).

In some embodiments, the present disclosure employs a rejection interval to improve the performance of the trained model, wherein a rejection occurs when the classifier confidence score is close to its decision boundary, and the sample is rejected for exceptional handling, such as rescan or manual inspection. In some embodiments, the rejection interval is defined by two thresholds with respect to the estimated posteriori probability of each class. The posteriori probabilities of being sensitive can be estimated using a parametric form of a sigmoid:

$$P ( y = + 1 \vert f ) = \frac{1}{1 + exp \{ - ( Af + B ) \}}$$

where f is the classification score, and A and B are the sigmoid parameters that have to be estimated based on the training set. Parameters A and B are estimated by minimizing the cross-entropy loss function between the true posterior and the estimated posterior. Let the true label of the n-th sample be \tilde{y}⁽ⁿ⁾∈{−1,+1}, then the target true posterior probability is

$${t}^{( n )} = \frac{{\tilde{y}}^{( n )} + 1}{2} .$$

If the size of the training dataset is N, then the goal is to minimize the cross-entropy loss on all the couples {t⁽ⁿ⁾,f⁽ⁿ⁾}ₙ₌₁^{N}.

In some embodiments, the rejection interval may be defined by determining two thresholds. By performing validation on training set, the thresholds are selected to reject a pre-defined amount of data. Those thresholds can be used for rejecting test samples, but they can also be used to eliminate low confidence samples in the training set, in order to retrain the classifier on high confidence data only.

Using machine learning classifier for binary classification, a multi-dimensional decision boundary is built, and the classifier determined the category of the sample based on this boundary. Due to the biological variability of the bacterial samples the “distance” of the samples from the boundary is different, which make the decision of the classifiers with different confidence levels. In order to improve the classification performances of the classifiers, an error-rejection strategy, (known also as high/low-confidence decisions in the clinical diagnostic literature) was used. Since most of the misclassified samples lie near the multi-dimensional decision boundary and thus are identified with high risk of being misclassified. Using this approach the system will not classify these samples (lie near the multi-dimensional decision boundary), with the risk-tolerance being a controllable parameter, and the result is a lowering of the risk of misclassification.

In some embodiments, at step 110, a trained machine learning model of the present disclosure may be applied to target spectral data obtained from a target sample, to predict a susceptibility of bacteria in the sample to one or more specified therapies.

### Experimental Results

### Infrared Absorption Spectra of UTI Bacteria

The present inventors studied 1005 different bacterial isolates derived directly from urine samples of UTIs patients, as follows:

567 isolates of E. coli,

220 isolates of Klebsiella pneumonia,

121 isolates of Pseudomonas aeruginosa, and

97 isolates of other UTI bacteria (Acinetobac baumannii, Citrobacter koseri, Enterobacter aerogenes, Enterobacter cloacae, Enterococcus cloacae Asbriae, Enterococcus faecium, Enterococcus faecalis, Enterococcus Spp, Klebsiella oxytoca, Klebsiella Spp, Morganella morganii, Pantoea Spp, Proteus mirabilis, Providencia stuartii, Serratia marcescens, Staphylococcus aureus, Staphylococcus saprophyticus, Streptococcus agalactiae).

These isolates were identified at a species level as well as their known susceptibility to most common used antibiotics, using the classical methods MALDI-TOF and VITEK2, respectively.

The samples where then processed for spectrometric measurements, by purifying the infecting bacteria directly from the urine as described herein above. A subset consisting of ten E. coli isolates was randomly selected, as detailed in table 1.

TABLE 1
Bacterial susceptibility Categories labels (sensitive (S)/resistant (R)) with respect
to six different antibiotics of ten of the E. coli isolates, selected randomly.

| Isolate numbers as | Antibiotics | | | | | |
|---|---|---|---|---|---|---|
| they appear in the | | | | | Sulfamethoxa | Amoxicillin |
| medical files | Ampicillin | Cefuroxime | Ceftriaxone | Ciprofloxacin | Trimeth | ClavulA |
| 306382 | S | S | S | S | S | S |
| 306358 | S | S | S | S | S | S |
| 306365 | R | S | S | S | R | I |
| 306366 | R | R | R | R | S | I |
| 307201 | R | S | S | R | R | S |
| 307212 | S | S | S | S | R | S |
| 307234 | R | S | S | S | R | S |
| 307402 | R | S | S | S | S | I |
| 307395 | R | S | S | R | R | S |
| 307368 | S | S | S | S | S | S |

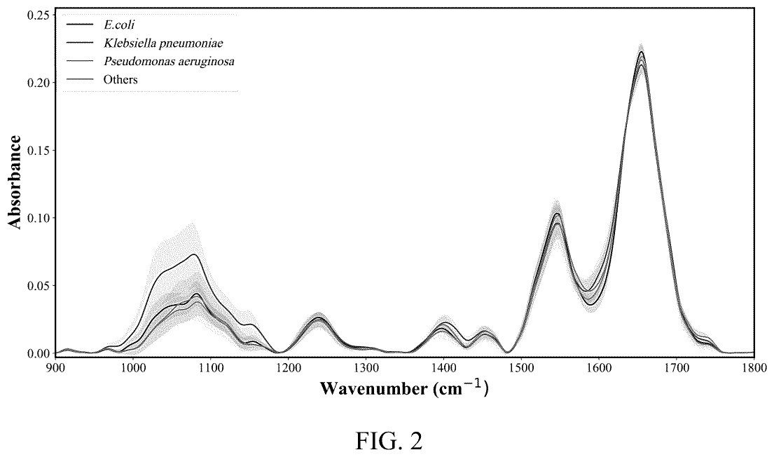

FIG. 2 shows the average IR absorption spectra of E. coli, Klebsiella pneumonia, Pseudomonas aeruginosa and other UTI bacteria in the 900-1800 cm⁻¹ region. As can be seen in FIG. 2, all the absorption features that represent the biomolecules that comprise the examined bacterial samples (e.g., proteins, lipids, nucleic acids and carbohydrates) appear in the spectra. Proteins contribute mainly in the 1480-1727 cm⁻¹ wavenumber region. The main contributors of the absorption bands centered at 1402 cm⁻¹ are fatty acids (C═O symmetric stretching of COO⁻ group), while carbohydrates are the main contributors to the absorption bands in the 900-1200 cm⁻¹ wavenumber region (C—O—C, C—O dominated by ring vibrations in various polysaccharides). Nucleic acids contribute mainly to the absorption bands centered at ˜1079 cm⁻¹ (P═O symmetric stretching in DNA, RNA and phospholipids).

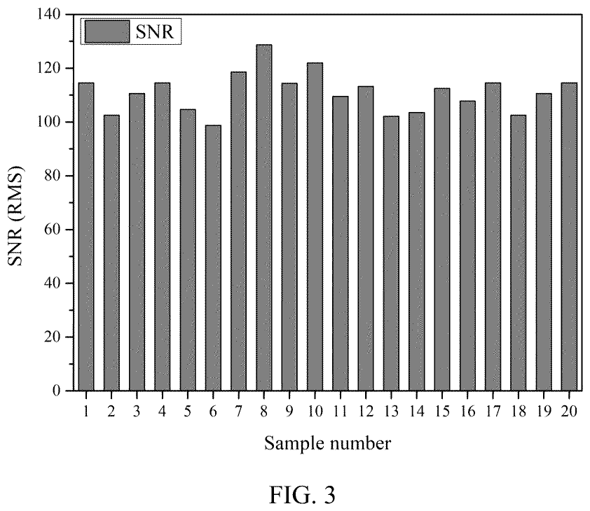

A bacterial isolate acquires resistance to specific antibiotic due to small mutations in its genome, thus the spectral changes among resistant and sensitive isolates are very small. Therefore, it is highly important to prepare the samples in an adequate manner, to acquire high SNR spectra with highly reproducible measurements, to enable classification with reasonable accuracy. FIG. 3 shows the calculated SNR of 20 different isolates. It can be seen that the SNR is ˜100, which is relatively high.

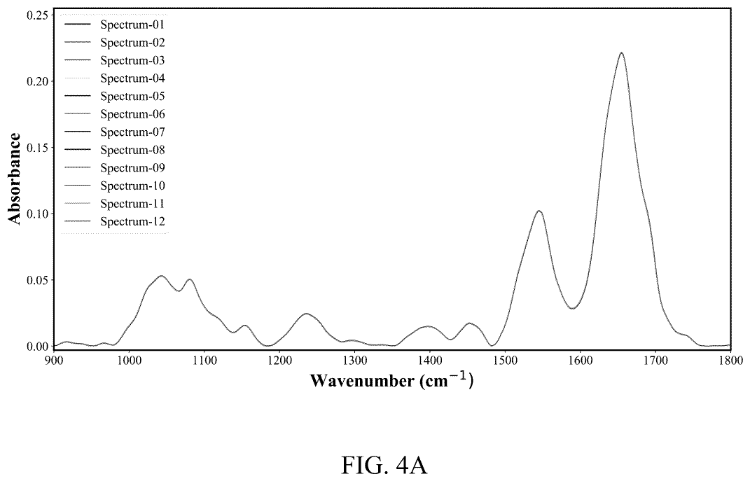

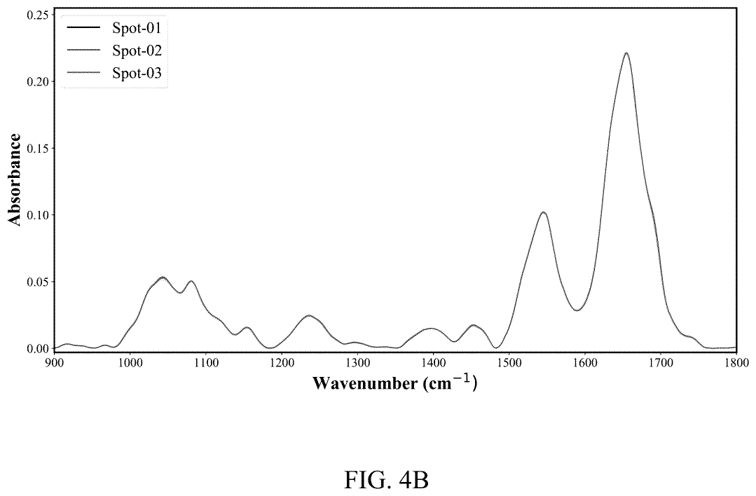

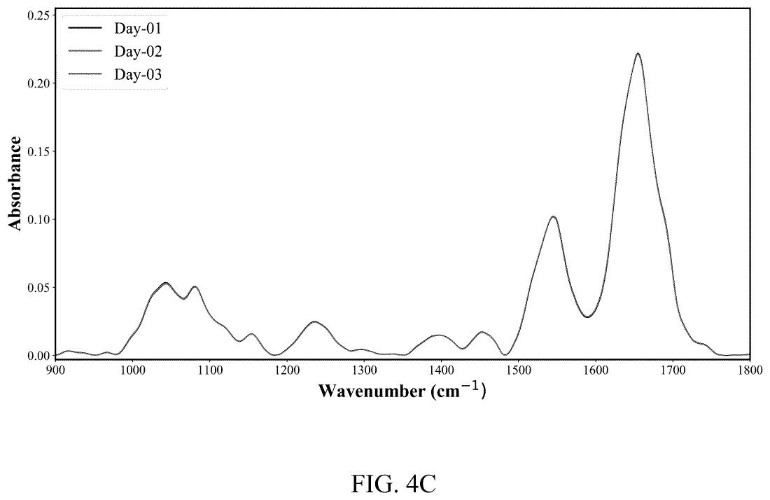

In order to verify reproducibility of results, 12 spectra were measured from different sites of the same sample of each of the investigated isolates. As an example, 12 spectra of one E. coli isolate, acquired from different sites of the same sample, are presented in FIG. 4A in the 900-1800 cm⁻¹ after preprocessing. The spectra are overlaid each other, demonstrating the high reproducibility of the spectra. FIG. 4B shows the averages of three infrared spectra of the same isolate from three different preparations (spots). FIG. 4C shows the averages of three infrared spectra of the same isolate measured from the same spot at three different days.

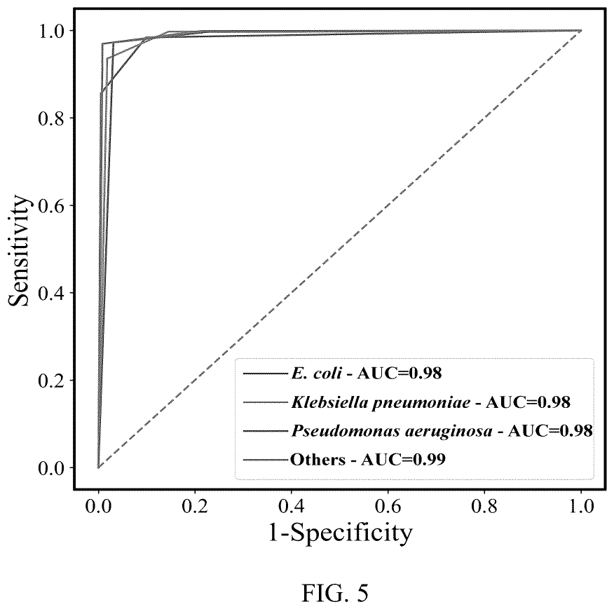

The different bacteria E. coli, Klebsiella pneumoniae, Pseudomonas aeruginosa and other UTI bacteria are similar and overlap with each other (FIG. 2), thus, a quadratic SVM (qSVM) classifier was used for taxonomic classification. The receiver-operating characteristic (ROC) curves of the classifier qSVM for the classification among E. coli, Klebsiella pneumonia, Pseudomonas aeruginosa and other UTI bacteria are presented in FIG. 5. The performance of the qSVM classifier is commonly presented as the area under the curve (AUC) of the ROC.

The performances of the qSVM classifier for the classification among, E. coli, Klebsiella pneumoniae, Pseudomonas aeruginosa and others UTI bacteria are summarized in Table 2 as confusion matrices. The calculated success rate was 97%.

TABLE 2
Confusion matrices of the classification among E. coli, Klebsiella pneumonia,
Pseudomonas aeruginosa and other UTI bacteria. The classification
was performed using XGBoost classifier based on infrared absorption spectra
in the 900-1800 cm⁻¹ region. Errors are calculated as standard
deviations of the performances.

| | | Predicted | | | |
|---|---|---|---|---|---|
| | | | Klebsiella | Pseudomonas | |
| | | E. coli | pneumonia | aeruginosa | others |
| True | E. coli | 554 (97.71%) | 8 (1.41%) | 4 (0.71%) | 1 (0.18%) |
| | Klebsiella | 2 (0.91%) | 214 (97.27%) | 2 (0.91%) | 2 (0.91%) |
| | pneumonia | | | | |
| | Pseudomonas | 2 (1.65%) | 1 (0.83%) | 115 (95.04%) | 3 (2.48%) |
| | aeruginosa | | | | |
| | others | 1 (1.03%) | 1 (1.03%) | 0 (0.00%) | 95 (97.94%) |

XGBoost

### Bacterial Susceptibility to Antibiotics

The present inventors then used selected features of the second derivative spectra in the 900-1800 cm⁻¹ as an interim analysis for the classification between the different categories, which was found to allow better bacterial susceptibility discrimination. The task is one of a binary classification of the spectra of each of the examined bacterial isolates of E. coli, Klebsiella pneumonia and Pseudomonas aeruginosa that were grouped based on susceptibility, to a specific antibiotic, as resistant or sensitive.

E. coli

The susceptibility of the E. coli isolates were determined with respect to Amoxicillin, Ampicillin, Ceftazidime, Ceftriaxone, Cefuroxime, Cefuroxime-Axetil, Cephalexin, Ciprofloxacin, Gentamicin, Nitrofurantoin, Piperacill-Tazobactam and Sulfamethoxa-Trimeth.

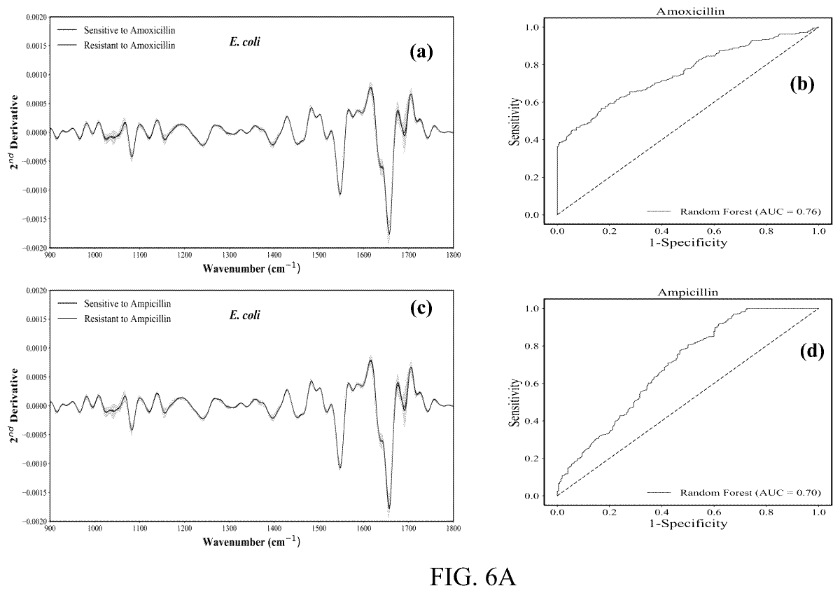

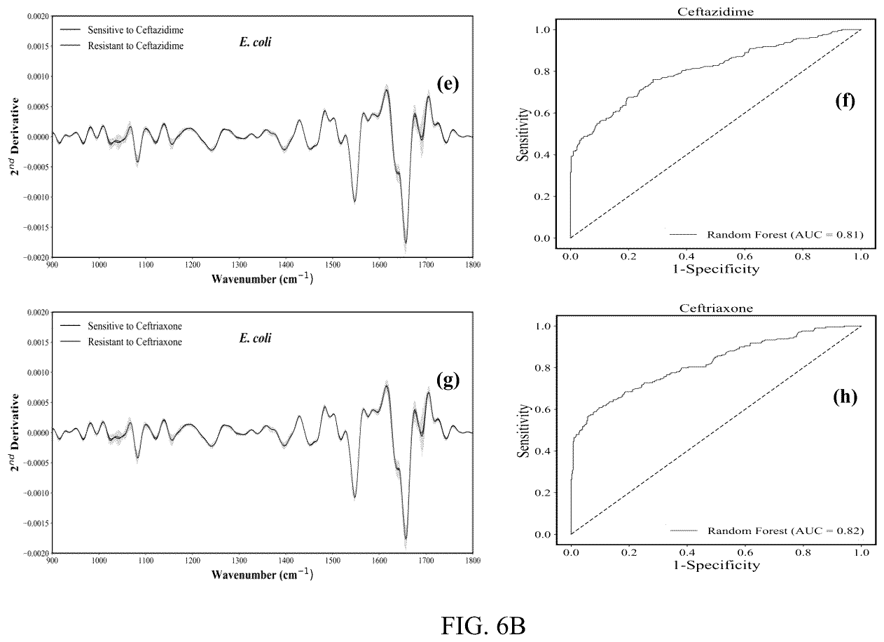

FIGS. 6A-6B present the average second derivative IR spectra of E. coli, in the 900-1800 cm⁻¹ region grouped as sensitive or resistant to: Amoxicillin (panel a), Ampicillin (panel c), Ceftazidime (panel e), and Ceftriaxone (panel g). The ROC curves of the classification of these antibiotics are respectively presented in FIGS. 6A-6B, panels (b), (d), (f) and (h). Results were also obtained (not shown) for Cefuroxime, Cefuroxime-Axetil, Cephalexin, Ciprofloxacin, Gentamicin, Nitrofurantoin, Piperacill-Tazobactam and Sulfamethoxa-Trimeth.

Several classifiers were examined, and an RF classifier was selected as providing best classification performances. The performances of the RF classifier for the classification between E. coli isolates sensitive and resistant to the tested antibiotics are summarized in Table 3. Two different experiments were performed; in the first experiment a classification threshold was defined and the classifier determined the category of the sample based on this threshold. Due to the biological variability of the bacterial samples, the “distance” of the samples from the threshold is different, which results in variations in confidence variables among the classifiers. Thus, in the second experiment, and in order to improve the classification performances of the classifiers, an error-rejection strategy was applied, so that low confidence decisions (for samples with scores close to the threshold) are rejected.

TABLE 3
Performances of RF classifier for classifying between the E. coli isolates as sensitive or resistant
to 12 different antibiotics, using feature selection of the second derivative spectra.

| | Rejected | | | | | | | | |
|---|---|---|---|---|---|---|---|---|---|
| | interval (%) | S | R | AUC | Acc | SE | SP | PPV | NPV |
| Amoxicillin | 0 | 397 | 170 | 0.76 | 0.70 ± | 0.78 ± | 0.50 ± | 0.79 ± | 0.50 ± |
| | | | | | 0.11 | 0.15 | 0.13 | 0.15 | 0.15 |
| | 15 | | | 0.77 | 0.72 ± | 0.81 ± | 0.52 ± | 0.80 ± | 0.54 ± |
| | | | | | 0.11 | 0.20 | 0.11 | 0.11 | 0.10 |
| Ampicillin | 0 | 181 | 384 | 0.70 | 0.64 ± | 0.55 ± | 0.68 ± | 0.45 ± | 0.76 ± |
| | | | | | 0.14 | 0.18 | 0.12 | 0.15 | 0.13 |
| | 14 | | | 0.75 | 0.66 ± | 0.64 ± | 0.67 ± | 0.48 ± | 0.80 ± |
| | | | | | 0.11 | 0.17 | 0.09 | 0.14 | 0.11 |
| Ceftazidime | 0 | 408 | 159 | 0.81 | 0.75 ± | 0.81 ± | 0.60 ± | 0.84 ± | 0.55 ± |
| | | | | | 0.11 | 0.18 | 0.15 | 0.18 | 0.16 |
| | 15 | | | 0.84 | 0.75 ± | 0.74 ± | 0.79 ± | 0.90 ± | 0.54 ± |
| | | | | | 0.12 | 0.13 | 0.11 | 0.14 | 0.12 |
| Ceftriaxone | 0 | 407 | 160 | 0.82 | 0.75 ± | 0.80 ± | 0.62 ± | 0.84 ± | 0.55 ± |
| | | | | | 0.16 | 0.14 | 0.14 | 0.13 | 0.17 |
| | 9 | | | 0.82 | 0.75 ± | 0.83 ± | 0.54 ± | 0.82 ± | 0.56 ± |
| | | | | | 0.11 | 0.14 | 0.15 | 0.15 | 0.13 |
| Cefuroxime- | 0 | 351 | 190 | 0.77 | 0.71 ± | 0.79 ± | 0.55 ± | 0.76 ± | 0.59 ± |
| Axetil | | | | | 0.10 | 0.17 | 0.14 | 0.15 | 0.14 |
| | 14 | | | 0.79 | 0.72 ± | 0.76 ± | 0.64 ± | 0.79 ± | 0.59 ± |
| | | | | | 0.08 | 0.16 | 0.14 | 0.18 | 0.16 |
| Cefuroxime | 0 | 371 | 196 | 0.78 | 0.73 ± | 0.82 ± | 0.55 ± | 0.77 ± | 0.62 ± |
| | | | | | 0.10 | 0.18 | 0.14 | 0.18 | 0.12 |
| | 15 | | | 0.80 | 0.75 ± | 0.81 ± | 0.63 ± | 0.81 ± | 0.64 ± |
| | | | | | 0.13 | 0.17 | 0.14 | 0.13 | 0.18 |
| Cephalexin | 0 | 372 | 167 | 0.79 | 0.74 ± | 0.81 ± | 0.57 ± | 0.81 ± | 0.58 ± |
| | | | | | 0.11 | 0.17 | 0.14 | 0.16 | 0.11 |
| | 9 | | | 0.79 | 0.73 ± | 0.78 ± | 0.61 ± | 0.82 ± | 0.56 ± |
| | | | | | 0.13 | 0.15 | 0.12 | 0.13 | 0.14 |
| Ciprofloxacin | 0 | 405 | 161 | 0.85 | 0.79 ± | 0.87 ± | 0.60 ± | 0.85 ± | 0.64 ± |
| | | | | | 0.14 | 0.14 | 0.09 | 0.15 | 0.10 |
| | 15 | | | 0.88 | 0.81 ± | 0.89 ± | 0.60 ± | 0.85 ± | 0.69 ± |
| | | | | | 0.10 | 0.15 | 0.16 | 0.13 | 0.16 |
| Gentamicin | 0 | 498 | 68 | 0.86 | 0.82 ± | 0.86 ± | 0.58 ± | 0.94 ± | 0.35 ± |
| | | | | | 0.13 | 0.15 | 0.16 | 0.14 | 0.15 |
| | 15 | | | 0.82 | 0.81 ± | 0.84 ± | 0.6 ± | 0.94 ± | 0.34 ± |
| | | | | | 0.16 | 0.16 | 0.13 | 0.17 | 0.13 |
| Nitrofurantoin | 0 | 502 | 47 | 0.90 | 0.89 ± | 0.91 ± | 0.62 ± | 0.96 ± | 0.40 ± |
| | | | | | 0.13 | 0.17 | 0.15 | 0.14 | 0.13 |
| | 14 | | | 0.85 | 0.86 ± | 0.88 ± | 0.62 ± | 0.96 ± | 0.33 ± |
| | | | | | 0.15 | 0.15 | 0.12 | 0.16 | 0.13 |
| Piperacill- | 0 | 506 | 58 | 0.88 | 0.85 ± | 0.85 ± | 0.81 ± | 0.97 ± | 0.39 ± |
| Tazobactam | | | | | 0.17 | 0.16 | 0.14 | 0.17 | 0.12 |
| | 12 | | | 0.86 | 0.84 ± | 0.84 ± | 0.8 ± | 0.98 ± | 0.32 ± |
| | | | | | 0.13 | 0.15 | 0.13 | 0.18 | 0.16 |
| Sulfamethoxa- | 0 | 371 | 194 | 0.75 | 0.70 ± | 0.77 ± | 0.57 ± | 0.77 ± | 0.56 ± |
| Trimeth | | | | | 0.12 | 0.13 | 0.11 | 0.15 | 0.13 |
| | 14 | | | 0.78 | 0.71 ± | 0.81 ± | 0.51 ± | 0.76 ± | 0.59 ± |
| | | | | | 0.10 | 0.16 | 0.15 | 0.16 | 0.12 |

Klebsiella pneumonia

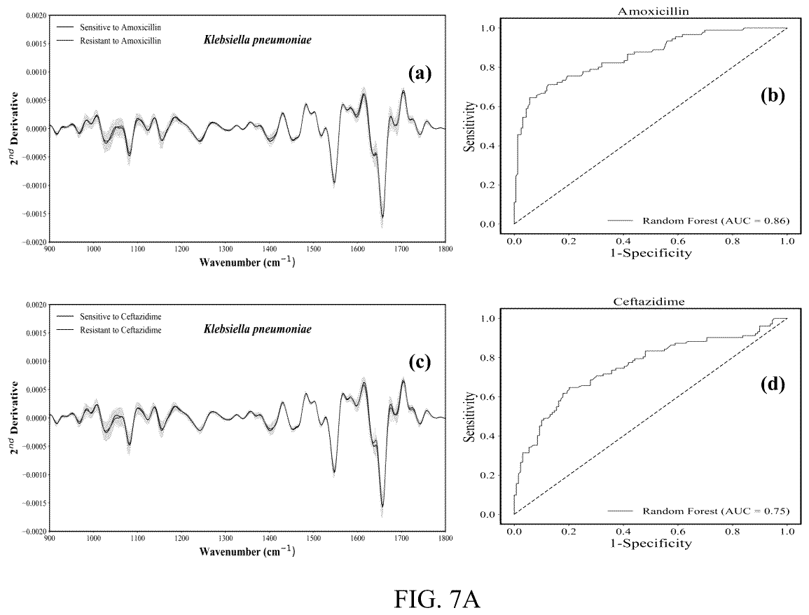

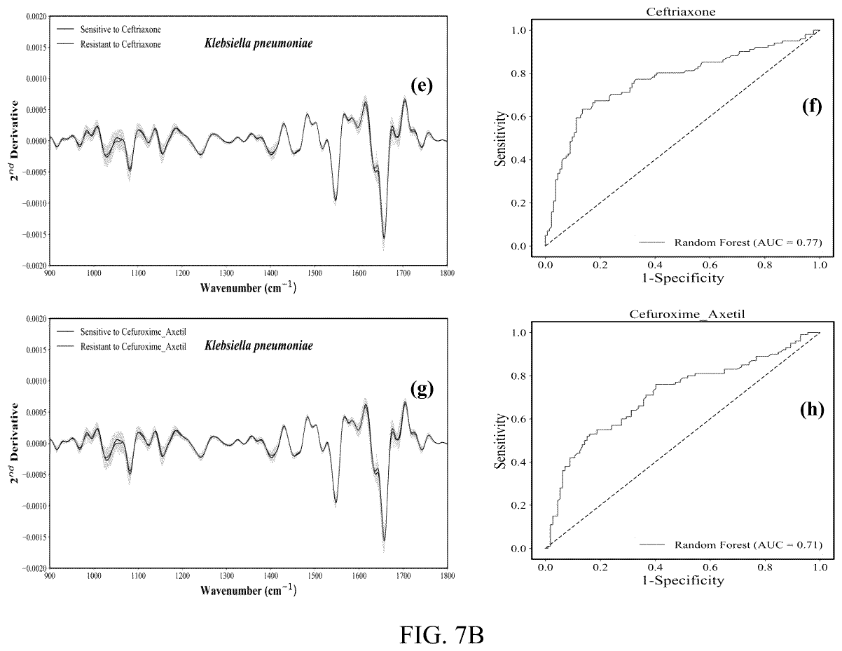

The susceptibility of the Klebsiella pneumonia isolates was determined with respect to Amoxicillin, Ceftazidime, Cefbriaxone, Cefuroxime, Cefuroxime-Axetil, Cephalexin, Ciprofloxacin, Gentamicin, Nitrofuirantoin, Piperacill-Tazobactam and Sulfamethoxa-Trimeth. FIGS. 7A-7B present the average second derivative IR spectra of Klebsiella pneumonia, in the 900-1800 cm⁻¹ region grouped as sensitive of resistant to: Amoxicillin (panel a), Ceftazidime (panel c), Ceftriaxone (panel e) and Cefuroxime (panel g). The ROC curves of the classification of these antibiotics are respectively presented in panels (b), (d), (f) and (h). Results were also obtained for the Cefuroxime-Axetil, Cephalexin, Ciprofloxacin, and Gentamicin, and Nitrofurantoin, Piperacill-Tazobactam and Sulfamethoxa-Trimeth, respectively (not shown). The performances of the RF classifier for the classification between Klebsiella pneumonia isolates sensitive and resistant to the tested antibiotics are summarized in Table 4 similar to E. coli (Table 3).

TABLE 4
Performances of RF classifier for classifying between the Klebsiella pneumoniae isolates as sensitive
or resistant to 11 different antibiotics. Using feature selection of the second derivative spectra.

| | Rejected | | | | | | | | |
|---|---|---|---|---|---|---|---|---|---|
| | interval | | | | | | | | |
| | (%) | S | R | AUC | Acc | SE | SP | PPV | NPV |
| Amoxicillin | 0 | 159 | 61 | 0.86 | 0.78 ± | 0.82 ± | 0.68 ± | 0.87 ± | 0.59 ± |
| | | | | | 0.17 | 0.16 | 0.15 | 0.14 | 0.13 |
| | 14 | | | 0.89 | 0.82 ± | 0.86 ± | 0.71 ± | 0.88 ± | 0.66 ± |
| | | | | | 0.13 | 0.18 | 0.19 | 0.15 | 0.13 |
| Ceftazidime | 0 | 129 | 91 | 0.75 | 0.71 ± | 0.65 ± | 0.80 ± | 0.82 ± | 0.61 ± |
| | | | | | 0.14 | 0.16 | 0.14 | 0.11 | 0.12 |
| | 14 | | | 0.77 | 0.74 ± | 0.74 ± | 0.75 ± | 0.80 ± | 0.67 ± |
| | | | | | 0.15 | 0.19 | 0.14 | 0.13 | 0.14 |
| Ceftriaxone | 0 | 133 | 87 | 0.77 | 0.73 ± | 0.77 ± | 0.67 ± | 0.78 ± | 0.66 ± |
| | | | | | 0.17 | 0.14 | 0.17 | 0.14 | 0.15 |
| | 4 | | | 0.77 | 0.74 ± | 0.76 ± | 0.70 ± | 0.79 ± | 0.66 ± |
| | | | | | 0.18 | 0.18 | 0.15 | 0.12 | 0.14 |
| Cefuroxime-Axetil | 0 | 112 | 95 | 0.71 | 0.69 ± | 0.76 ± | 0.60 ± | 0.69 ± | 0.68 ± |
| | | | | | 0.13 | 0.15 | 0.16 | 0.09 | 0.12 |
| | 14 | | | 0.73 | 0.69 ± | 0.84 ± | 0.51 ± | 0.67 ± | 0.72 ± |
| | | | | | 0.13 | 0.17 | 0.16 | 0.13 | 0.14 |
| Cefuroxime | 0 | 121 | 99 | 0.66 | 0.62 ± | 0.62 ± | 0.62 ± | 0.67 ± | 0.57 ± |
| | | | | | 0.14 | 0.19 | 0.15 | 0.12 | 0.14 |
| | 14 | | | 0.68 | 0.64 ± | 0.71 ± | 0.55 ± | 0.66 ± | 0.61 ± |
| | | | | | 0.15 | 0.17 | 0.18 | 0.09 | 0.16 |
| Cephalexin | 0 | 121 | 90 | 0.71 | 0.69 ± | 0.64 ± | 0.77 ± | 0.79 ± | 0.61 ± |
| | | | | | 0.15 | 0.16 | 0.15 | 0.13 | 0.15 |
| | 11 | | | 0.73 | 0.70 ± | 0.68 ± | 0.73 ± | 0.77 ± | 0.63 ± |
| | | | | | 0.15 | 0.16 | 0.18 | 0.08 | 0.10 |
| Ciprofloxacin | 0 | 161 | 59 | 0.87 | 0.83 ± | 0.84 ± | 0.78 ± | 0.91 ± | 0.65 ± |
| | | | | | 0.17 | 0.17 | 0.15 | 0.16 | 0.13 |
| | 5 | | | 0.88 | 0.83 ± | 0.84 ± | 0.82 ± | 0.93 ± | 0.65 ± |
| | | | | | 0.17 | 0.16 | 0.14 | 0.14 | 0.13 |
| Gentamicin | 0 | 179 | 41 | 0.91 | 0.86 ± | 0.87 ± | 0.86 ± | 0.96 ± | 0.59 ± |
| | | | | | 0.17 | 0.17 | 0.16 | 0.14 | 0.13 |
| | 8 | | | 0.92 | 0.87 ± | 0.85 ± | 0.93 ± | 0.98 ± | 0.59 ± |
| | | | | | 0.15 | 0.13 | 0.20 | 0.12 | 0.20 |
| Nitrofurantoin | 0 | 62 | 150 | 0.80 | 0.75 ± | 0.58 ± | 0.82 ± | 0.57 ± | 0.82 ± |
| | | | | | 0.17 | 0.19 | 0.19 | 0.16 | 0.17 |
| | 13 | | | 0.84 | 0.78 ± | 0.87 ± | 0.74 ± | 0.58 ± | 0.93 ± |
| | | | | | 0.15 | 0.12 | 0.14 | 0.14 | 0.14 |
| Piperacill- | 0 | 179 | 41 | 0.90 | 0.85 ± | 0.85 ± | 0.83 ± | 0.96 ± | 0.56 ± |
| Tazobactam | | | | | 0.16 | 0.18 | 0.15 | 0.14 | 0.14 |
| | 12 | | | 0.93 | 0.88 ± | 0.85 ± | 0.99 ± | 1.00 ± | 0.61 ± |
| | | | | | 0.17 | 0.16 | 0.17 | 0.11 | 0.11 |
| Sulfamethoxa- | 0 | 126 | 92 | 0.70 | 0.67 ± | 0.70 ± | 0.63 ± | 0.72 ± | 0.60 ± |
| Trimeth | | | | | 0.16 | 0.13 | 0.10 | 0.13 | 0.11 |
| | 11 | | | 0.71 | 0.67 ± | 0.70 ± | 0.65 ± | 0.73 ± | 0.61 ± |
| | | | | | 0.17 | 0.20 | 0.15 | 0.15 | 0.13 |

Pseudomonas aeruginosa

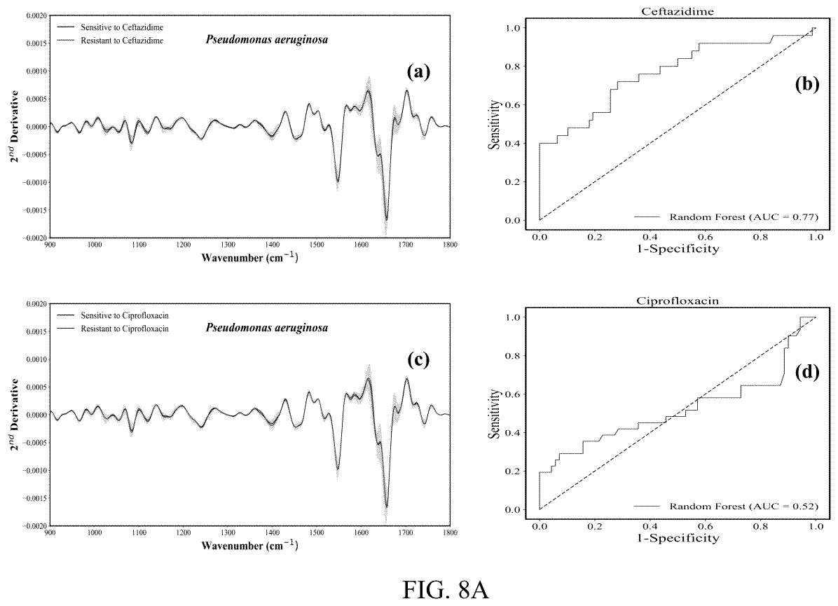

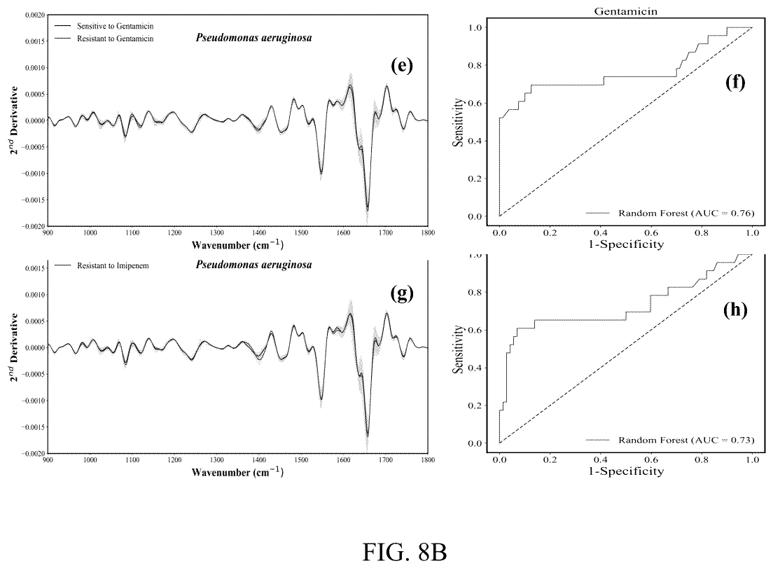

The susceptibility of the Pseudomonas aeruginosa isolates was determined with respect to Ceftazidime, Ciprofloxacin, Gentamicin, Imipenem, Levofloxacin, Meropenem, Piperacill-Tazobactam, Piperacillin and Tobramycin. FIGS. 8A-8B present the average second derivative IR spectra of Pseudomonas aeruginosa, in the 900-1800 cm⁻¹ region grouped as sensitive of resistant to: Ceftazidime (panel a), Ciprofloxacin (panel c), Gentamicin (panel e), and Imipenem (panel g). The ROC curves of the classification of these antibiotics are respectively presented in panels (b), (d), (f) and (h). Results were also obtained for Levofloxacin, Meropenem, Piperacill-Tazobactam, and Piperacillin, and Tobramycin respectively (not shown). The performances of the RF classifier for the classification between Pseudomonas aeruginosa isolates sensitive and resistant to the tested antibiotics are summarized in Table 5 similar to E. coli (Table 3).

TABLE 5
Performances of RF classifier for classifying between the Pseudomonas aeruginosa
isolates as sensitive or resistant to 9 different antibiotics. Using
feature selection of the second derivative spectra.

| | Rejected | | | | | | | | |
|---|---|---|---|---|---|---|---|---|---|
| | interval (%) | S | R | AUC | Acc | SE | SP | PPV | NPV |
| Ceftazidime | 0 | 78 | 19 | 0.77 | 0.77 ± | 0.84 ± | 0.50 ± | 0.87 ± | 0.43 ± |
| | | | | | 0.16 | 0.16 | 0.16 | 0.13 | 0.12 |
| | 12 | | | 0.77 | 0.74 ± | 0.8 ± | 0.5 ± | 0.87 ± | 0.38 ± |
| | | | | | 0.11 | 0.15 | 0.20 | 0.14 | 0.12 |
| Ciprofloxacin | 0 | 70 | 27 | 0.52 | 0.54 ± | 0.58 ± | 0.43 ± | 0.72 ± | 0.28 ± |
| | | | | | 0.20 | 0.16 | 0.18 | 0.13 | 0.12 |
| | 6 | | | 0.53 | 0.54 ± | 0.63 ± | 0.29 ± | 0.70 ± | 0.24 ± |
| | | | | | 0.19 | 0.18 | 0.14 | 0.16 | 0.16 |
| Gentamicin | 0 | 80 | 17 | 0.76 | 0.73 ± | 0.70 ± | 0.88 ± | 0.96 ± | 0.38 ± |
| | | | | | 0.15 | 0.18 | 0.14 | 0.14 | 0.13 |
| | 2 | | | 0.74 | 0.7 ± | 0.67 ± | 0.88 ± | 0.96 ± | 0.36 ± |
| | | | | | 0.16 | 0.14 | 0.19 | 0.13 | 0.13 |
| Imipenem | 0 | 72 | 18 | 0.73 | 0.69 ± | 0.65 ± | 0.86 ± | 0.95 ± | 0.38 ± |
| | | | | | 0.17 | 0.17 | 0.17 | 0.13 | 0.15 |
| | 4 | | | 0.68 | 0.67 ± | 0.74 ± | 0.4 ± | 0.83 ± | 0.28 ± |
| | | | | | 0.13 | 0.17 | 0.14 | 0.13 | 0.14 |
| Levofloxacin | 0 | 63 | 33 | 0.56 | 0.59 ± | 0.64 ± | 0.51 ± | 0.71 ± | 0.42 ± |
| | | | | | 0.12 | 0.13 | 0.18 | 0.12 | 0.14 |
| | 13 | | | 0.57 | 0.56 ± | 0.63 ± | 0.44 ± | 0.68 ± | 0.38 ± |
| | | | | | 0.14 | 0.14 | 0.18 | 0.12 | 0.12 |
| Meropenem | 0 | 77 | 20 | 0.64 | 0.63 ± | 0.69 ± | 0.40 ± | 0.82 ± | 0.25 ± |
| | | | | | 0.14 | 0.17 | 0.15 | 0.15 | 0.13 |
| | 15 | | | 0.66 | 0.66 ± | 0.72 ± | 0.44 ± | 0.83 ± | 0.29 ± |
| | | | | | 0.15 | 0.16 | 0.18 | 0.14 | 0.13 |
| Piperacill- | 0 | 76 | 10 | 0.91 | 0.93 ± | 0.94 ± | 0.86 ± | 0.98 ± | 0.66 ± |
| Tazobactam | | | | | 0.18 | 0.14 | 0.18 | 0.13 | 0.14 |
| | 9 | | | 0.93 | 0.93 ± | 0.93 ± | 0.93 ± | 0.99 ± | 0.65 ± |
| | | | | | 0.12 | 0.18 | 0.15 | 0.15 | 0.15 |
| Piperacillin | 0 | 59 | 23 | 0.72 | 0.72 ± | 0.74 ± | 0.68 ± | 0.86 ± | 0.50 ± |
| | | | | | 0.14 | 0.14 | 0.21 | 0.11 | 0.14 |
| | 11 | | | 0.72 | 0.71 ± | 0.73 ± | 0.67 ± | 0.85 ± | 0.49 ± |
| | | | | | 0.15 | 0.18 | 0.14 | 0.11 | 0.18 |
| Tobramycin | 0 | 79 | 15 | 0.78 | 0.75 ± | 0.76 ± | 0.66 ± | 0.92 ± | 0.34 ± |
| | | | | | 0.13 | 0.18 | 0.16 | 0.10 | 0.12 |
| | 4 | | | 0.80 | 0.75 ± | 0.76 ± | 0.69 ± | 0.93 ± | 0.36 ± |
| | | | | | 0.14 | 0.16 | 0.14 | 0.13 | 0.13 |

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The description of a numerical range should be considered to have specifically disclosed all the possible subranges as well as individual numerical values within that range. For example, description of a range from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A system comprising:
at least one hardware processor; and
a non-transitory computer-readable storage medium having stored thereon program instructions, the program instructions executable by the at least one hardware processor to:
receive by a trained Machine Learning (ML) model, target spectral data associated with a target sample of a bodily fluid obtained from a target subject, wherein the bodily fluid is selected from a plurality of bodily fluids, each associated with spectral data; and
estimate a response in said target subject to each specified therapy in a set of specified therapies, based on the received target spectral data and the target sample of bodily fluid.

2. The system according to claim 1 wherein the trained ML model is produced by:
receiving said spectral data associated with samples of each of the plurality of bodily fluid obtained from a corresponding plurality of subjects having a specified type of infectious disease,
receiving data identifying a response parameter to one or more of the set of therapies associated with each of said subjects, and
training a machine learning model on a training set comprising:
(i) said spectral data associated with each of said plurality of bodily fluid samples, and
(ii) labels associated with said response parameters.

3. The system of claim 1, wherein, with respect to each of said bodily fluid samples, said spectral data is acquired less than 5 hours from a time of obtaining of said bodily fluid sample.

4. The system of claim 2, wherein at least one of said plurality of bodily fluid samples and said target sample are each a urine sample, and said specified type of infectious disease is urinary tract infection (UTI).

5. The system of claim 1, wherein said spectral data is acquired from bacteria obtained from each of said bodily fluid samples.

6. The system of claim 5, wherein said spectral data represents infrared (IR) absorption in said bacteria.

7. The system of claim 1, wherein said spectral data is within the wavenumber range of 600-4000 cm⁻¹.

8. The system of claim 1, wherein said set of specified therapies comprises one or more antibiotics.

9. The system of claim 2, wherein said response parameter is one of: sensitive and resistant.

10. The system of claim 1, wherein said bodily fluids comprise one of: whole blood, blood plasma, blood serum, lymph, urine, saliva, semen, synovial fluid, and spinal fluid.

11. The system of claim 1, wherein said program instructions are further executable to perform one of: feature manipulations and dimensionality reduction with respect to said spectral data.

12. The system of claim 2, wherein, with respect to said training set, said spectral data associated with each of said plurality of bodily fluid samples are labeled with said labels.

13. The system of claim 2, wherein said training set further comprises, with respect to at least some of said subjects, labels associated with clinical data.

14. A method comprising:
receiving by a trained Machine Learning (ML) model, target spectral data associated with a target sample of a bodily fluid obtained from a target subject, wherein the bodily fluid is selected from a plurality of bodily fluids, each associated with spectral data; and
estimate a response in said target subject to each specified therapy in a set of specified therapies, based on the received target spectral data and the target sample of bodily fluid.

15. The method of claim 14 wherein the trained ML model is produced by:
receiving said spectral data associated with samples of each of the plurality of bodily fluid obtained from a corresponding plurality of subjects having a specified type of infectious disease,
receiving data identifying a response parameter to one or more of the set of specified therapies associated with each of said subjects, and
training a machine learning model on a training set comprising:
(i) said spectral data associated with each of said plurality of bodily fluid samples, and
(ii) labels associated with said response parameters.

16. The method of claim 14, wherein, with respect to each of said bodily fluid samples, said spectral data is acquired less than 5 hours from a time of obtaining of said bodily fluid sample.

17. The method of claim 14, wherein at least one of said plurality of bodily fluid samples and said target sample are each a urine sample, and said specified type of infectious disease is urinary tract infection (UTI).

18. The method of claim 14, wherein said spectral data is acquired from bacteria obtained from each of said bodily fluid samples, and wherein said spectral data represents infrared (IR) absorption in said bacteria.

19-26. (canceled)

27. A computer program product comprising a non-transitory computer-readable storage medium having program instructions embodied therewith, the program instructions executable by at least one hardware processor to:
receive by a trained Machine Learning (ML) model, target spectral data associated with a target sample of a bodily fluid obtained from a target subject, wherein the bodily fluid is selected from a plurality of bodily fluids, each associated with spectral data; and
estimate a response in said target subject to each specified therapy in a set of specified therapies, based on the received target spectral data and the target sample of bodily fluid.

28. The computer program product according to claim 27 wherein the trained ML model is produced by:
receiving said spectral data associated with samples of each of the plurality of bodily fluid obtained from a corresponding plurality of subjects having a specified type of infectious disease,
receiving data identifying a response parameter to one or more of the set of therapies associated with each of said subjects, and
training a machine learning model on a training set comprising:
(i) said spectral data associated with each of said plurality of bodily fluid samples, and
(ii) labels associated with said response parameters.

29-39. (canceled)
